(19) United States
(12) Patent Application Publication
(10) Pub. No.: US 2007/0015983 A1
(43) Pub. Date: Jan. 18, 2007

(54) Method for controlling the range of functions of diagnostic devices

(76) Inventors: Karl Werner, Wiesloch (DE); Peter Blasberg, Weinheim (DE); Rudolf Supka, Heppenheim (DE)

Correspondence Address: Roche Diagnostics OPERATIONS Inc., 9115 Hague Road, PO Box 50457, Indianapolis, IN, 46250-0457, US

(21) Appl. No.: 11/351,619

(22) Filed: Feb. 10, 2006

(30) Foreign Application Priority Data
Feb. 10, 2005 (DE) 10 2005 006 111.7

(51) Int. Cl. A61B 5/00; A61B 5/05

(52) U.S. Cl. 600/347; 600/300

(57) ABSTRACT

In particular for the measurement of blood glucose concentrations, portable diagnostic devices are disclosed that are user-friendly and safe to handle. A method is proposed which controls the range of functions of diagnostic devices and avoids incorrect operation of diagnostic devices of this kind, by substantially preventing unintentional and unnoticed alteration of critical parameters, typically system time and/or system date. The method includes a key-recognition step and an enable step. The key-recognition step determines whether at least one key module is connected to the diagnostic device. Depending on whether such a connection exists, the alteration of at least one critical parameter stored and/or generated in the diagnostic device is permitted or prevented in the enable step. A diagnostic device on which the method according to the invention can be implemented in one of its embodiments is also proposed, as is a corresponding key module, and also a diagnostic system which comprises a diagnostic device and at least one key module.

### REFERENCE TO RELATED APPLICATIONS

The present application is based on and claims priority to DE Patent Application No. 10 2005 00611.7, filed Feb. 10, 2005, which is hereby incorporated by reference in its entirety.

### TECHNICAL FIELD

The invention relates to a method for controlling the range of functions of diagnostic devices, in particular a method for avoiding incorrect operations of measurement devices for determination of blood glucose concentrations. The invention further relates to a computer program for carrying out a method according to the invention. The invention further relates to a diagnostic device for carrying out the method according to the invention, and to the use of a key module for controlling the range of functions, in particular for avoiding incorrect operations of a diagnostic device according to the invention, and to a diagnostic system which comprises a diagnostic device according to the invention and at least one key module.

### BACKGROUND

The monitoring of blood glucose concentrations is an essential part of the daily routine of diabetics. The blood glucose concentration has to be determined quickly and simply several times a day in order, if appropriate, to be able to take suitable medical measures. So as not to restrict the diabetic's daily routine any more than is necessary, suitable portable devices are often employed which are intended to be easy to carry around and to operate, such that the blood glucose concentration can be measured, for example, at the workplace or even during leisure time.

Various portable devices are presently available on the market, some of them functioning according to different measurement methods. Various diagnostic methods are used in these devices, for example optical or even electrochemical measurement methods. An important element in these measurement methods often takes the form of corresponding test elements, in most cases test strips, for example electrochemical test strips. Typically, about 5 to 7 such test strips are needed each day by a diabetic. A problem which often arises here is that the properties of the test strips used may vary from batch to batch. This leads to in some cases huge variations in the accuracy of the blood glucose concentration measurement. In many known commercial systems, batch information relating to the test strips therefore has to be input into a diagnostic device (for example a blood glucose meter) prior to a corresponding blood glucose concentration measurement, so that the diagnostic device can correctly “interpret” the measurement data of the test strip by means of this batch information, in order to determine a correct blood glucose concentration from said data. There are several known methods for inputting the batch information into the analysis module. For example, it is possible here to use suitable barcodes which, for example, can be arranged on a packaging of the test strips and which can be read by the diagnostic device. Alternatively, systems are also known in which a data carrier, often also called a ROM key (see for example DE 692 33 262 T2), is attached to the test strip container holding the test strips. This data carrier can, for example, be attached loosely to a test strip container used for storing the test strips and, when the test strips are used, it can then be inserted for example into a suitable receiving slit of a diagnostic device.

In monitoring a diabetic's blood glucose concentration, it is of essential importance that measured values of the blood glucose concentration are recorded as a function of time. In this way, a patient or a treating physician can use the time profile of the blood glucose concentration to establish an optimum medication, in particular an optimum dosage of insulin, and to set up a suitable medication timetable. The time profile of the blood glucose concentration can also be used to establish any unsuitable aspects of the patient's approach, in particular unsuitable habits, for example the wrong diet. A suitably adapted diet can accordingly be planned.

To permit this functionality of recording a time profile of the blood glucose concentration, many commercially available diagnostic devices, in particular blood glucose concentration meters, are designed such that measured values can be recorded together with a corresponding measurement time. This measurement time generally comprises a date and a clock time and is in most cases stored together with the measured value in a data memory of the diagnostic device. This information can then either be evaluated directly on the diagnostic device and, for example, shown in graph form, or said information can also be transferred to another system, for example a computer system. This information can then be analyzed, for example on a computer (e.g. a laptop or PDA (personal digital assistant)), for example by the blood glucose concentration being shown in graph form as a function of time. This evaluation can be performed by the patient himself or also by a treating physician. To evaluate the information and therefore optimize the therapy, commercial products are available, in particular software products, which make it easier to analyze the information.

For the evaluation of the blood glucose concentration profile, coordination of measured values to measurement times is therefore almost indispensable. The settings on the diagnostic devices, in particular also the setting of date and clock time, are generally made via buttons, for example by menu control. However, since diagnostic devices carried by the patient should be small, handy and easy to use, the number of operating elements, in particular buttons, is in most cases kept to a minimum in such devices. Portable and inexpensive devices often have only 2 to 3 buttons. The corresponding menu-controlled setting of the parameters, in particular of the date and clock time, is therefore relatively complex despite the menu control. Particularly when used by elderly persons or also by children, it may therefore happen in many cases that, as a result of incorrect operation, in particular accidental actuation of buttons, the date and/or clock time of the diagnostic device are accidentally altered, and this often goes unnoticed. This incorrect operation is made all the more probable by the fact that, in many menu controls, all the parameters are set in a linear sequence, no distinction being made between critical, one-off parameter settings (e.g. date, clock time) and non-critical parameter settings, for example the patient's name or the like. Critical and non-critical parameters are also in most cases not saved separately. By means of an incorrect operation of the kind described, in particular by accidental and unnoticed alteration of time and date, information kept over a long period of time may become largely worthless for therapy optimization.

In the prior art, similar problems and corresponding solutions are known from other technical fields. For example, in the mobile phone sector it is known that undesired actuation of the buttons of a mobile phone can be avoided by the whole control panel being blocked. Entries are made possible again only after a specific combination of buttons has been actuated. As soon as the control panel is freed, however, it is generally possible for all the parameters to be altered again (in most cases by menu control), so that, when this principle is transferred to diagnostic devices, critical parameters could once again be wrongly set. Moreover, direct transfer of this principle, known from mobile telephony, to diagnostic devices which in most cases have just a few buttons is not directly possible.

Moreover, from the field of operating systems for computer systems, it is known to make a safety inquiry before altering specially critical parameters. Only after explicit confirmation by the user that the critical parameter is actually to be altered is said parameter altered. However, transferring this principle to diagnostic devices would considerably reduce the user-friendliness of the operation of such diagnostic devices and would considerably increase the time required for an entry to be made. In particular, elderly persons or children, or patients with inadequate language abilities, could be confused by this additional inquiry and the associated more complex menu control, and this could lead to further incorrect operations.

Therefore, the object of the present invention is to make available a method and a system which at least largely avoids the stated disadvantages of the prior art. In particular, the method and the system are intended to greatly reduce the risk of incorrect operation of diagnostic devices through undesired and unnoticed adjustment of critical parameters and to permit simple use of diagnostic devices of this kind.

### SUMMARY

This object is achieved by the invention having the features of the independent claims. Advantageous developments of the invention are characterized in the dependent claims. The wording of all the claims is herewith made by reference part of the content of this description.

A method is provided for controlling the range of functions of diagnostic devices, typically for avoiding incorrect operations of measurement devices for determination of blood glucose concentrations. In addition to measurement devices for determination of blood glucose concentrations, however, other diagnostic devices are also considered here in which the method according to the invention can be advantageously applied in one of its embodiments. This refers, for example, to devices for measuring blood lipid concentrations or also to devices for measuring blood pressure or body temperature. In particular, the expression diagnostic device is to be understood in a broad sense here, so that, in addition to device for medical diagnosis, the method can also be applied to devices for chemical diagnosis, for example for measuring pH values of liquids or for measuring concentrations of pollutants.

The method comprises the steps described below, although these do not necessarily have to be carried out in the stated sequence. Besides the method steps specified, additional method steps not described here may also be carried out. Moreover, method steps can also be carried out in parallel.

The method first comprises a key-recognition step which determines whether at least one key module is connected to the diagnostic device. The method also comprises an enable step in which, depending on the key-recognition step, the alteration of at least one critical parameter stored and/or generated in the diagnostic device is permitted or prevented. In particular, it is advantageous if the method is carried out such that an alteration of the at least one critical parameter is prevented in the enable step if it is found, in the key-recognition step, that the at least one key module is connected to the diagnostic device.

A basic concept of the method according to the invention is therefore that of using a diagnostic system which comprises, in addition to a diagnostic device, at least one key module. The diagnostic device determines whether the key module is connected to the diagnostic device. Two possibilities then exist in principle. If the key module is connected to the diagnostic device, the diagnostic device can then permit or prevent the alteration of the at least one critical parameter. Conversely, when the key module is disengaged from the diagnostic device, the alteration of the at least one critical parameter is accordingly prevented or permitted. The connection of the at least one key module to the diagnostic device thus acts like the actuation of an enabling switch.

In practice, it has proven particularly advantageous if the enablement, that is to say the permission to alter the at least one critical parameter, is realized through disengagement of the key module from the diagnostic device. This is particularly advantageous when the at least one key module comprises at least one data storage element on which at least one batch information item of at least one test element is stored. When the at least one key module is connected to the diagnostic device, the latter can transfer at least one batch information item from the key module into at least one data memory of the diagnostic device. This configuration is therefore particularly advantageous because many diagnostic devices, as has been described above, work with test elements, for example test strips, whose properties vary from batch to batch. To solve this problem, as has also been described above, a so-called ROM key is in many cases used which, during use of the test elements, is connected to the diagnostic device, for example inserted into a corresponding slit, and the batch-specific information items are transferred from the ROM key into the diagnostic device. One such ROM key is typically attached to or provided with each new batch of test elements acquired by the patient. This ROM key can now be used at the same time to carry out the method according to the invention. The diagnostic device can be configured, for example, in such a way that critical parameters cannot be altered when the ROM key is connected to the diagnostic device. Only when the ROM key is removed from the diagnostic device, which requires a conscious effort on the part of the user, can the alteration be permitted.

The diagnostic device can include various types of parameters, which in particular can be stored in the diagnostic device. Thus, a distinction can be made in particular between critical and non-critical parameters. Thus, the method can be configured in particular in such a way that only certain, in particular some or all of the critical parameters are enabled to be altered by the user through the above-described enable step. Non-critical parameters can typically be altered at any time and independently of the connection of the key module to the diagnostic device. In particular, the at least one critical parameter can comprise a time parameter and/or a date parameter. It is thus possible, in particular, to prevent a situation where, during operation of the diagnostic device, a system time and/or a system date is accidentally altered and this alteration often goes unnoticed, as a result of which, as has been described above, many measurements would become unusable for later analysis. By using the method according to the invention, the user-friendliness of the diagnostic device is thus considerably increased and the risk of incorrect operations, in particular during use by elderly patients or children, is greatly reduced.

Moreover, a diagnostic device is proposed for carrying out a method according to the invention in accordance with one of the above-described embodiments. In particular, this diagnostic device, as has been described above, can comprise a measurement device for determination of blood glucose concentrations. As has also been described above, however, other diagnostic devices are also conceivable. The diagnostic device comprises means for carrying out a diagnostic method, in particular for determining blood glucose concentrations. As has been described above, these means for carrying out the diagnostic method can, for example, comprise means for reading out (e.g., evaluating or analyzing) a test element, in particular a test strip, typically an electrochemical test strip. These means can in particular involve an electronic device by means of which it is possible to carry out current/voltage measurements between electrodes of the test element and/or capacitive measurements between these electrodes. Electronic devices of this kind are known from commercial measurement devices, for example commercial measurement devices for blood glucose concentration measurement. However, as has been described above, the invention is not restricted to electrochemical measurement devices but can also be used, for example, for optical measurement methods.

The diagnostic device according to the invention also comprises means for connecting the diagnostic device to at least one key module. These connecting means can be configured in different ways, the term “connecting” having to be understood in a broad sense. In particular, the means for connecting the diagnostic device to a key module can comprise mechanical and/or electrical elements. For example, these means can comprise a slit or a recess in a housing of the diagnostic device, into which the at least one key module is inserted when connecting the key module to the diagnostic device. For example, as has been described above, this can be the slit in a ROM key for a blood glucose concentration meter.

Moreover, these means for connecting the diagnostic device to a key module can, for example, also comprise an interface, such that, when the at least one key module is connected to the diagnostic device, data or energy can also be exchanged between the diagnostic device and the key module. This is particularly advantageous in combination with a design of the diagnostic device in which this diagnostic device also comprises at least one data memory and means for transferring at least one batch information item of at least one test element into the at least one data memory of the diagnostic device. The abovementioned interface in this case represents a constituent part of the means for transferring this at least one batch information item. The data memory can, for example, be a volatile or also a non-volatile data memory in the diagnostic device.

Since however, as has been described above, the term “connecting” is to be understood in a broad sense, any other kind of connection between the diagnostic device and the at least one key module is also conceivable in principle according to the invention. In particular, this can also involve a wireless connection, for example an infrared interface or a high-frequency connection. In this case, the at least one key module is connected to the diagnostic device by the at least one key module being brought towards the diagnostic device, for example by being brought to a predetermined minimum distance therefrom or into a specified spatial position.

Moreover, the diagnostic device comprises means for generating and/or storing the at least one critical parameter. These means for storing the at least one critical parameter can, for example, involve the at least one data memory already discussed above, or, for example, also a separate data memory. These means for storing the at least one critical parameter can also comprise a volatile and/or also a non-volatile memory. In particular, if several critical parameters are used, these critical parameters can also be stored in different data memories. These means for storing the at least one critical parameter can, for example, involve the data memory already described above for storing the at least one batch information item, or also a separate data memory. In particular, the diagnostic device can also comprise at least one computer, typically a microcomputer. The data memory or data memories can then be a constituent part of this computer or can also be configured as separate electronic memory elements.

Moreover, the diagnostic device comprises means for determining whether at least one key module is connected to the diagnostic device. The configuration of these means for determining the connection generally depends on the configuration of the above-described means for connecting the diagnostic device to the at least one key module. Thus, the execution of the above-described key-recognition step also depends on the configuration of these means. For example, the detection of the at least one key module can take place by purely mechanical means. In particular, when the at least one key module is connected to the diagnostic device, a (mechanical or electromechanical) switch can be actuated, it being possible to infer from the latter's switching status whether the at least one key module is connected to the diagnostic device. Moreover, these means for determining whether the at least one key module is connected to the diagnostic device can also comprise electrical or electronic means. For example, these means, as has already-been described above, can comprise an interface. When connecting the at least one key module to the diagnostic device, it is then possible to determine, by means of a method known to the skilled person, whether the at least one key module is connected to the interface. Such methods are often used in computer technology and are based, for example, on measuring the potential and/or capacitance between two electrical contacts of the interface of the diagnostic device. For example, if an electric bridge is created between the two electrodes by connection of the at least one key module to the interface, this can then be detected in the manner described. Moreover, the determination of whether the at least one key module is connected to the diagnostic device can also be done by inquiring whether information stored on the key module for the diagnostic device is available to be read.

However, other configurations of means for determining whether the at least one key module is connected to the diagnostic device are also conceivable. Thus, for example, optical devices may be considered, for example light barriers which detect such a connection. In the above-described case in which the connection consists in bringing the at least one key module and the diagnostic device close to one another, the means for determining whether the at least one key module is connected to the diagnostic device can, for example, comprise a transmitting device for emission of electromagnetic radiation. It is thus possible to detect, for example, whether emitted electromagnetic radiation is absorbed by a key module located in the vicinity. This principle is used, for example, in transponder technology or in the form of magnetic strip security labels in shops.

Moreover, if the diagnostic device comprises at least one computer (see above), the at least one computer can also form a constituent part of the means for determining whether the at least one key module is connected to the diagnostic device.

Moreover, the diagnostic device comprises means for altering at least one critical parameter depending on the connection between the diagnostic device and the at least one key module. In particular, these means for altering the at least one critical parameter can comprise input elements, for example input buttons on a surface of a housing of the diagnostic device. Other input means are also conceivable, for example a keypad, a touchscreen, an interface to an input unit and/or to a computer, or a voice data input. Moreover, the means for altering the at least one critical parameter depending on the connection of the at least one key module to the diagnostic device can comprise an electronic logic circuit. As has been described above, a user is given the possibility of altering the at least one critical parameter only when the preset enable condition is satisfied, that is to say when the at least one key module is or is not connected to the diagnostic device. Alternatively or in addition to an electronic logic circuit, a computer can also be used, for example a computer on which a program for menu control can be run. In this case, the menu control, in particular the menu-controlled alteration of the at least one critical parameter, is configured by corresponding configuration of the software in such a way that the alteration of the at least one critical parameter is possible only when the above-described enable condition is satisfied. This way of implementing the method according to the invention has in particular the advantage that already existing measurement appliances can be taken over without any substantial change to their hardware, and only the software of these measurement appliances has to be slightly modified. In the case of the use of existing measurement appliances in particular, a device according to the invention is to be understood as one such measurement appliance with software that is stored in this measurement appliance and/or can be run on this measurement appliance.

The invention moreover proposes the use of a key module for avoiding incorrect operations of diagnostic devices, particularly of the diagnostic device according to the invention in accordance with one of the configurations described above. This key module comprises means for connecting the key module to a diagnostic device according to the invention. As has already been described above, these means can be either mechanical means for producing a mechanical connection, or electrical means. For example, these means for connecting the key module to the diagnostic device can comprise an interface, in particular for example an interface with one or more electrode contacts, or for example a wireless interface, for example an infrared interface. If, as has been described above, a wireless connection between the key module and the diagnostic device is used, the means for connecting the key module to the diagnostic device can, for example, also comprise a transponder. Moreover, the means for connecting the key module to the diagnostic device can, for example, also comprise mechanical devices which, for example, permit insertion of the key module into the diagnostic device. For example, the key module can comprise a housing which, by being suitably configured, can be inserted particularly easily into a corresponding slit or a corresponding recess of complementary design in a housing of the diagnostic device.

The key module also comprises signal means making it possible to determine whether the key module is connected to the diagnostic device. As has been described above, the configuration of these signal means depends on the nature of the connection between the key module and the diagnostic device. Thus, for example, they can be mechanical signal means which, for example, actuate a switch upon connection to the diagnostic device, or electrical and/or optical signal means. For example, as has been described above, these signal means can comprise an interface which is connected to a corresponding interface of the diagnostic device upon connection of the key module to the diagnostic device. The connection can then be determined, for example, using a method described above. Batch information items which relate to at least one test element, for example a test strip, and which can be stored in at least one data storage element in the key module, can then be transferred for example to the diagnostic device via this interface. Alternatively or in addition to this, the key module can also comprise further means for transferring the at least one batch information item into at least one data memory of the diagnostic device. In particular, the above-described data storage element of the key module can comprise a non-volatile memory (in particular a ROM).

According to the invention, a diagnostic system is also proposed, typically a system for determination of blood glucose concentrations. This diagnostic system comprises a diagnostic device according to the invention in accordance with one of the above-described configurations. The diagnostic system moreover comprises at least one key module in accordance with one of the above-described configurations.

The method according to the invention, the diagnostic device according to the invention, the key module and the diagnostic system according to the invention have numerous and important advantages over corresponding devices and methods known from the prior art. As has been described above, these advantages in particular concern user-friendliness, since incorrect operations, particularly by elderly persons or children, are avoided and/or the risk of such incorrect operations is greatly reduced. Thus, the risk of critical patient data, in particular measured values, being wrongly evaluated or interpreted is also greatly reduced. Dangerous misdiagnosis is thus to a large extent avoided. There is also less risk of measured data being rendered unusable despite the patient carrying out the measurements carefully, for example because of information on the times of these measurements being lost. This also further reduces the risk of misdiagnosis and largely ensures a chronologically unbroken diagnosis.

The scope of the invention also includes a computer program which, when run on a computer or computer network, executes the key-recognition step and the enable step of the method according to the invention. In particular, this computer program can comprise program code means. In particular, these program code means can be stored on a computer-readable data carrier. The scope of the invention also includes a data carrier on which a data structure is stored which, after it has been loaded into a user memory and/or main memory of a computer or computer network of a diagnostic device according to the invention, can execute the key-recognition step and the enable step of the method according to the invention in one configuration. In particular, the scope of the invention also includes a corresponding computer program product with program code means stored on a machine-readable carrier. Under computer program product, the program is here to be understood as a commercially available product. It can in principle be present in any desired form, e.g. on paper or a computer-readable data carrier and can in particular be sent via a data transmission network.

Further details and features of the invention will become evident from the following description of preferred illustrative embodiments in conjunction with the dependent claims. The respective features can be realized either singly or in combination with one another. The invention is not limited to the illustrative embodiments. The illustrative embodiments are shown schematically in the figures. Identical reference numbers in the individual figures designate identical elements or elements which are of identical function or which correspond to one another in terms of their function.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic design plan of an illustrative embodiment of a diagnostic system according to the invention for determination of blood glucose concentrations; and

FIG. 2 shows a schematic flow chart of an illustrative embodiment of a method according to the invention for avoiding incorrect operations of diagnostic devices.

Skilled artisans appreciate that elements in the figures are illustrated for simplicity and clarity and have not necessarily been drawn to scale. For example, the dimensions of some of the elements in the figure may be exaggerated relative to other elements to help improve understanding of the embodiment(s) of the present invention.

In order that the invention may be more readily understood, reference is made to the following examples, which are intended to illustrate the invention, but not limit the scope therof.

### DETAILED DESCRIPTION

The following description of the preferred embodiment is merely exemplary in nature and is in no way intended to limit the invention or its application or uses.

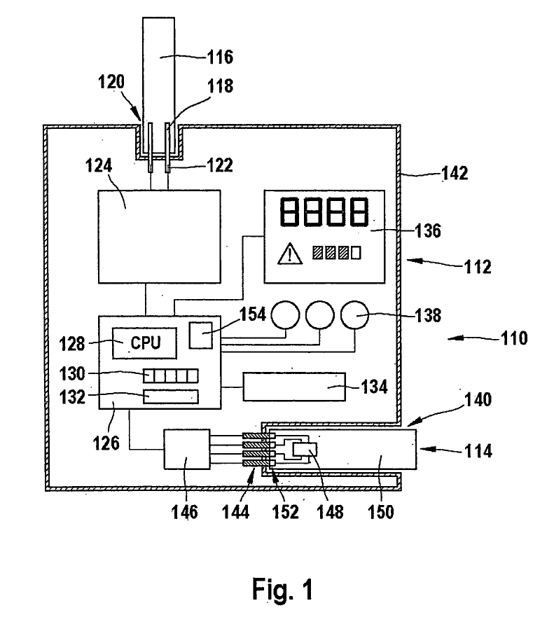

FIG. 1 depicts an illustrative embodiment of a diagnostic system 110 according to the invention for determination of blood glucose concentrations. The system 110 comprises a diagnostic device in the form of a portable blood glucose meter 112 and a key module in the form of a ROM key 114.

The portable blood glucose meter 112 in this illustrative embodiment is designed as a measurement device for determining the blood glucose concentration by means of a test strip 116. In this illustrative embodiment, the test strip 116 is an electrochemical test strip of the type described above, having two measurement electrodes 118 which are coated with enzymes and mediators and to which a quantity of blood can be applied via a capillary system (not shown). In the view according to FIG. 1, the test strip 116 is pushed into a test strip receiver 120 in which the measurement electrodes 118 of the test strip 116 are contacted by corresponding electrodes 122 of the portable blood glucose meter 112. The electrodes 122 are connected to measurement electronics 124 via which the above-described measurement, for example a current/voltage measurement or a capacitive measurement, can be carried out in order then, for example, to be able to deduce a blood glucose concentration from a defined charge carrier concentration at the measurement electrodes 118. Such systems are known to the skilled person and are commercially available. The measurement electronics 124 are connected to a microcomputer 126. The microcomputer 126 comprises a processor unit 128 (indicated by CPU in FIG. 1) and also a volatile memory 130 and a non-volatile memory 132. In addition, the microcomputer 126 is also connected to a non-volatile memory unit 134 in which larger amounts of data can be stored, for example blood glucose concentration values stored over quite a long measurement period.

The microcomputer 126 is also connected to a display element 136, for example a liquid-crystal display. This liquid-crystal display 136 can be configured for example as a segmented display or also as a pixelated display (shown symbolically in FIG. 1 as a segmented display). The microcomputer 126 is also connected to operating elements 138 in the form of buttons, via which a user for example can start a measurement or can also alter parameters. In this illustrative embodiment, three operating elements 138 are provided.

The portable blood glucose meter 112 also comprises a ROM key receiver 140 which in this illustrative embodiment is configured as a slit in a housing 142 of the portable blood glucose meter 112. An interface 144 in the form of several electrodes is provided in this ROM key receiver 140 and is connected to the microcomputer 126 via an interface control 146.

In the view according to FIG. 1, the ROM key 114 has been pushed into the ROM key receiver 140 and comprises a data storage element in the form of a non-volatile memory (ROM) 148. This non-volatile memory 148 is fixed on a support plate 150 made of plastic and is connected to an interface 152 on the ROM key. In the state shown in FIG. 1, in which the ROM key 114 is inserted into the ROM key receiver 140, the interface 152 on the ROM key is connected to the interface 144 of the portable blood glucose meter 112.

In this illustrative embodiment, the microcomputer 126 plays a central role in the portable blood glucose meter 112. The microcomputer 126 on the one hand controls the blood glucose concentration measurement by means of the measurement electronics 124 in which measurements are started and corresponding measured values generated by the measurement electronics 124 are processed. For this purpose, these measured values, which are transmitted for example in the form of electronic signals to the microcomputer 126, are converted by the processor unit 128 into corresponding blood glucose concentrations. For this purpose, algorithms and parameters in particular, for example variables, can be used which can in particular be stored in the volatile memory 130 and in the non-volatile memory 132 of the microcomputer 126. The microcomputer 126 also comprises a clock 154. In this way, the blood glucose concentration values can be stored together with a measurement time and/or a measurement date, for example in the external, non-volatile memory unit 134. The system time or system date generated by the clock 154 thus represents a critical parameter which is in particular used later for the evaluation of the measured values.

The measurement data which are stored in one of the memories 130, 132 or 134 can also be managed by the microcomputer 126. For this purpose, the microcomputer 126 can, for example, execute a program for data management. The microcomputer 126 can also show, on the display element 136, the corresponding measurement data, for example the blood glucose concentrations from a measurement that has just been carried out. In more modern blood glucose meters 112, this can be displayed not just in the form of purely numerical values but also in particular as already graphically processed or partially processed information items, for example a time profile of the blood glucose concentrations over a defined period of time. Further information items too can be shown on the display element 136, for example a battery status display of a power supply (not shown in FIG. 1) of the portable blood glucose meter 112, or various warnings, for example a warning when the blood glucose concentration exceeds certain predetermined limit values.

A user operates the portable blood glucose meter 112 by way of the operating elements 138. Since only three operating elements 138 are provided in this illustrative embodiment, in particular for purposes of simplification and also for reasons of space, the blood glucose meter 112 can be controlled via a suitable menu. Menu-based controls of this kind are known to the skilled person and are commercially available. Thus, for example, a first operating element 138 could be used to select a menu option “Set system time/system date”, after which the same operating element or another operating element is then used to change the system time and/or the system date, for example by continuously increasing the time or date.

However, it is precisely in this way of operating the blood glucose meter 112 that operating errors can arise. Since elderly patients and children often have difficulties using a menu-based control, for example because of inability to read or because of lack of experience with menu-controlled applications of this kind, it may happen that the system time or system date is accidentally altered, without this being intended. Such alterations of the critical parameters “System time” and/or “System date” may, however, render the measurements completely unusable, especially if these alterations remain undiscovered for quite a long period of time. For this purpose, the microcomputer 126, the interface control 146 and the interface 144 of the blood glucose meter 112 in this illustrative embodiment are configured such that it is possible to determine whether a ROM key 114 has been pushed into the ROM key receiver 140 and accordingly connected to the blood glucose meter 112. This can be done, for example, by determining electrically, via the interface control 146, whether the electrode interface 144 is connected to the electrodes of the interface 152 of the ROM key 114. This can be done by a simple voltage measurement between two electrodes of the interface 144. Accordingly, the software of the microcomputer 126, which software can be stored for example in the volatile memory 130, contains a module element which asks (for example at regular intervals) whether the ROM key 114 is connected to the blood glucose meter 112.

The illustrative embodiment, shown in FIG. 1, of a system 110 according to the invention for determination of blood glucose concentration affords the advantage, in particular, that systems already in existence can be taken over without changes to the hardware or with only minor modifications to the hardware. In many cases, the only modification needed is to the software which is stored and run on the microcomputer 126. It should be noted in this context that, instead of a single microcomputer 126, it is also possible to use several computers, for example several interconnected microcomputers 126. It is especially advantageous if the described software module is configured in such a way that certain critical parameters can be altered only if the connection between the ROM key 114 and the blood glucose meter 112 is disengaged. The reasoning for this is in particular that, normally for a measurement, the ROM key 114 is connected permanently to the blood glucose meter 112, with batch information items being transferred once or repeatedly from the non-volatile memory 148 of the ROM key 114 via the interfaces 144 and 152 to the blood glucose meter 112, where they are stored in one or more of the memories 130, 132 and/or 134. These batch information items are required by the microcomputer 126 for interpretation of the measurement data of the test strip 116 which have been transmitted from the measurement electronics 124, so as to be able to generate correct results for the blood glucose concentration even when the properties of the test strip 116 fluctuate from batch to batch. Therefore, in this illustrative embodiment, the ROM key 114 in the first instance performs the function of a data carrier which is exchangeable and which is replaced by a new ROM key 114 when a new batch of test strips 116 is used. In addition, according to the invention, the ROM key 114 performs the function of a key module, with certain critical parameters being able to be altered by the user only when the ROM key 114 is disengaged from the blood glucose meter 112. An inadvertent and, in particular, unnoticed alteration to these critical parameters is thus almost impossible.

It should also be noted at this point that the expression “critical parameter” is also to be interpreted in a broad sense. In this illustrative embodiment shown in FIG. 1, the critical parameter described has been a system time and/or system date which can be altered by the user only when the ROM key 114 is disengaged from the blood glucose meter 112. Alternatively or in addition, however, an alteration of critical parameters can also be understood as other actions on the part of the user which are made accidentally and go unnoticed. For example, it is possible to imagine in particular preventing the inadvertent deletion of all the measured values that are stored in the non-volatile memory unit 134. It is also possible to prevent accidental alteration of critical limit values, for example limit values which, when they are exceeded, cause the patient to receive a visual or acoustic warning.

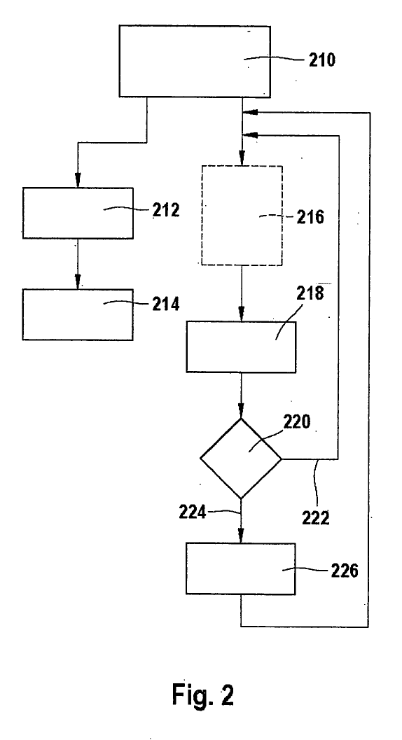

FIG. 2 shows a possible illustrative embodiment of a method according to the invention for avoiding incorrect operations of diagnostic devices, which method can be carried out, for example, with a system 110 according to the invention as shown in FIG. 1. In a first method step 210, a key module, in particular a ROM key 114 according to the view in FIG. 1, is connected to a diagnostic device, for example to a portable blood glucose meter 112 according to the view in FIG. 1. This connection can be made, for example, according to the illustrative embodiment described with reference to FIG. 1. In method step 212, batch-specific data are then transferred from the ROM key 114 to the blood glucose meter 112, in particular from the non-volatile memory 148 of the ROM key 114 into one or more of the memories 130, 132 and/or 134 of the blood glucose meter 112. Then, in method step 214, a measurement of the blood glucose concentration can be carried out by means of a test strip 116. It should also be noted at this point that the method steps shown do not necessarily have to be carried out in the specified sequence and that other method steps not shown in FIG. 2 can also be carried out.

In method step 216, after the connection of ROM key 114 and blood glucose meter 112, the user can optionally disengage both elements 112, 114 again, in particular by removing the ROM key 114 again from the ROM key receiver 140 of the blood glucose meter 112. According to the invention, the possibility for the user to alter certain critical parameters, such as the system time and/or system date, for example, is to be dependent on whether the ROM key 114 and the blood glucose meter 112 are actually connected or not. Accordingly, in method step 218, a key-recognition step is performed which checks whether a user has carried out method step 216 or not. The key-recognition step 218 thus checks whether the ROM key 114 is still connected to the blood glucose meter 112 or not. For this purpose, it is possible, for example, to use a simple control bit where, for example, as the outcome of the key-recognition step 218, the control bit is set to the value one when ROM key 114 and blood glucose meter 112 are connected, and is set to the value zero when ROM key 114 and blood glucose meter 112 are disengaged. For example, this control bit can be stored in the volatile memory 130 or in the non-volatile memory 132 of the microcomputer 126. An enable step 220 is then performed in which the outcome of the key-recognition step 218 is called up, for example by calling up said control bit. If it is found that ROM key 114 and blood glucose meter 112 are connected (step 222), then the procedure jumps back to before method step 216, i.e. an alteration of the critical parameters by the user is not permitted. However, non-critical parameters can be altered. The key-recognition step 218 is then performed again, so that it is possible to check continuously as to whether ROM key 114 and blood glucose meter 112 are connected.

If, however, it is found, in enable step 220, that ROM key 114 and blood glucose meter 112 are not (any longer) connected, i.e. that method step 216 has been carried out by the user, then (method step 224) method step 226 is carried out in which the alteration of critical parameters, in particular of a system time or a system date, by the user is permitted. It is then possible to jump back again to before method step 216, i.e. an interrogation is once again made, in method step 218, as to whether ROM key 114 and blood glucose meter 112 are connected. In this way, an interrogation can be made on a continuous basis.

The method shown by way of example in FIG. 2 can run in particular in the background, without a user being directly aware of this method being carried out. A user carries out the blood glucose concentration measurement (method step 214) independently of the method shown in FIG. 2 and uses the menu control of the blood glucose meter 112. It is only when the user attempts to alter parameters which are categorized as critical that the method shown in FIG. 2 makes itself noticed. In this case, this alteration of the critical parameter is possible only when method step 226 has been carried out, i.e. when alteration of this critical parameter has been explicitly permitted. This may be the case, for example, when said control bit shows the value zero. Alternatively, when, in a situation in which the alteration of the critical parameters is not permitted, the user nevertheless attempts to alter these, said user can be requested, by an image display, to first disengage the ROM key 114 from the blood glucose meter 112. This indication can also be provided with an explicit warning which tells the user that he is in the process of altering critical parameters, for example the system time and/or the system date.

The method shown in FIG. 2 can be loaded in particular as a single software module or in the form of several software modules into already existing blood glucose meters 112 and then run on these. In many cases, no adaptation of the hardware is necessary. The method shown is therefore inexpensive and easy to implement and it greatly reduces the risk of incorrect operation of a blood glucose meter 112.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modification and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A method for controlling the range of functions of a diagnostic device, the method comprising the following steps:
a) a key-recognition step which determines whether at least one key module is connected to the diagnostic device; and
b) an enable step in which, depending on the determination of the key-recognition step, the alteration of at least one critical parameter stored and/or generated in the diagnostic device is permitted or prevented.

2. The method of claim 1, wherein in the enable step, an alteration of the at least one critical parameter is prevented if it is found, in the key-recognition step, that the at least one key module is connected to the diagnostic device.

3. The method of claim 1, wherein at least two parameters are stored in the diagnostic device, at least one of these parameters comprising a critical parameter that can be altered depending on the insertion of the at least one key module, and in that at least one other of these parameters comprises a non-critical parameter that can be altered independently of the insertion of the at least one key module.

4. The method of claim 1, further comprising the following additional step:
c) from at least one data storage element of the at least one key module, at least one batch information item of at least one test element is transmitted into at least one data memory of the diagnostic device.

5. The method of claim 1, wherein at least one critical parameter comprises a time parameter and/or a date parameter.

6. A computer program comprising program code means for carrying out the method of claim 1, the computer program being adapted to run on a computer or computer network of a diagnostic device.

7. A computer-readable data carrier comprising the computer program of claim 6.

8. A data carrier or computer system on which a data structure is stored which, after loading into a user memory and/or main memory of a computer or computer network of a diagnostic device, runs the method of claim 1.

9. A computer program comprising program code means stored on a machine-readable carrier in order to carry out the method of claim 1 when the program is run on a computer or computer network of a diagnostic device.

10. A diagnostic device, comprising:
a) a means for carrying out a diagnostic method;
b) a means for connecting the diagnostic device to at least one key module;
c) a means for generating and/or storing at least one critical parameter;
d) a means for determining whether at least one key module is connected to the diagnostic device; and
e) a means for altering the at least one critical parameter depending on the connection of the at least one key module to the diagnostic device.

11. The diagnostic device of claim 10, wherein the means for carrying out a diagnostic method comprises a means for reading out a test element.

12. The diagnostic device of claim 11, wherein the means for reading out a test element comprises a means for reading out a test strip.

13. The diagnostic device of claim 10, further comprising:
f) at least one data memory; and
g) a means for transferring at least one batch information item of at least one test element into the at least one data memory.

14. The diagnostic device of claim 10, further comprising:
h) at least one computer.

15. A key module for controlling the range of functions of diagnostic devices, the key module comprising:
a) a means for connecting the key module to the diagnostic device of claim 10; and
b) a signal means making it possible to determine whether the key module is connected to the diagnostic device.

16. The key module of claim 15, further comprising:
c) at least one data storage element for storing at least one batch information item of at least one test element; and
d) a means for transferring the at least one batch information item into at least one data memory of the diagnostic device.

17. The key module of claim 16, wherein at least one data storage element comprises a non-volatile memory.

18. A diagnostic system, comprising:
a) the diagnostic device of claim 10; and
b) at least one key module comprising a means for connecting the key module to the diagnostic device and a signal means making it possible to determine whether the key module is connected to the diagnostic device.
